(19) United States
(12) Patent Application Publication
(10) Pub. No.: US 2023/0218801 A1
(43) Pub. Date: Jul. 13, 2023

(54) Bio-Material Composition and Methods of Use

(71) Applicant: Bone Solutions, Inc., Colleyville, TX (US)

(72) Inventors: Frankie L. Morris, Colleyville, TX (US); Brandon Roller, Colleyville, TX (US)

(21) Appl. No.: 18/000,215

(22) PCT Filed: May 27, 2021
(86) PCT No.: PCT/US21/34408

§ 371 Date: Nov. 29, 2022

Related U.S. Application Data
(60) Provisional application No. 63/032,847, filed on Jun. 1, 2020.

(51) Int. Cl. A61L 27/12; A61L 27/58; A61L 27/54; A61L 27/22; A61L 27/56

(52) U.S. Cl. CPC A61L 27/12; A61L 27/58; A61L 27/54; A61L 27/227; A61L 27/56; A61L 2430/02; A61L 2300/406; A61L 2300/232; A61L 2300/412

(57) ABSTRACT

The present disclosure relates to a bio-material composition comprising a dry potassium phosphate based mixture omprising: MgO, monobasic potassium phosphate, monobasic sodium phosphate, proteoglycans, calcium sodium phosphosilicate, and an antibiotic, wherein a weight percent ratio of monobasic potassium phosphate to MgO is between about 3:1 and 1:1, wherein the dry otassium phosphate based mixture is configured to be mixed with the aqueous solution to thereby form a reabsorbable bio-material slurry, wherein the proteoglycans are between about 1-10 weight percent of the dry composition, and wherein the proteoglycans act as active regulators of collagen fibrillogenesis to thereby structure tissue of a patient by organizing a bone extracellular matrix.

### TECHNICAL FIELD

This application claims priority to U.S. Provisional Application No. 63/032,847 entitled “Bio-Material Composition and Methods of Use,” filed on Jun. 1, 2020, the contents of which are hereby incorporated by reference in its entirety.

### TECHNICAL FIELD

The present disclosure relates to a bio-material composition and methods of use. Embodiments of the formed bio-material are osteoconductive and osteoinductive, thereby enabling new bone growth in the patient along a bone-implant interface as well as within the bone-implant interface.

### BACKGROUND

Unless otherwise indicated herein, the materials described in this section are not admitted to be prior art to the claims in this application.

Increasing numbers of sports, age, and trauma related injuries like broken bones, worn out joints, and torn ligaments have heightened the demand for bio-materials capable of treating orthopedic injuries. In response, companies have developed bone cements to attach various objects to bone, and bone fillers capable of treating bone fractures and other bone defects. There is also a need for a bio-material capable of stimulating bone formation and growth. Most existing bio-materials are made of calcium phosphates that promote significant new bone formation or relatively inert hardening polymers like polymethylmethcrylate (“PMMA”) that are poorly biocompatible.

U.S. Pat. No. 5,968,999 issued to Ramp et al, describes a PMMA based bone cement composition useful for orthopedic procedures. Unfortunately, PMMA-based bio- materials release considerable amounts of heat to the surrounding bone during the curing process causing cell death. The resulting materials shrink during setting and have poor resistance to fracture. PMMA biomaterials also possess slow rates of bio-absorption and poor bio-compatibility due to the release of a toxic monomer into the blood stream. There is little evidence that PMMA based materials promote any significant new bone formation.

A number of calcium phosphate based compositions have been developed as biomaterials in recent years. For example U.S. Pat. No. 6,331,312 issued to Lee et al., discloses an injectable calcium phosphate based composite useful as a bone filler and cement. The disclosed material is bio-resorbable and is designed for use in the repair and growth promotion of bone tissue as well as the attachment of screws, plates and other fixation devices. Lee's composition does not expand while setting and does not promote significant new bone formation. Many existing calcium phosphate based fillers and cements have high molar ratios of Ca to P making them poorly reabsorbable. Furthermore, a recent FDA release warns of serious complications from the use of existing calcium phosphate based bone fillers in treating fractures of the spine (FDA Public Health Web Notification, “Complications Related to the Use of Cement and Bone Void Fillers in Treating Compression Fractures of the Spine,” originally published Oct. 31, 2002, updated, May 27, 2004.)

### SUMMARY

The present disclosure comprises a bio-material composition and method of producing such, wherein one or more of the embodiments formed are osteoconductive and osteoinductive, thereby enabling new bone growth in the patient along a bone-implant interface as well as within the bone-implant interface.

In a first aspect, the present disclosure provides a bio-material composition comprising a dry potassium phosphate based mixture comprising: MgO, monobasic potassium phosphate, monobasic sodium phosphate, proteoglycans, calcium sodium phosphosilicate, and an antibiotic, wherein a weight percent ratio of monobasic potassium phosphate to MgO is between about 3:1 and 1:1, wherein the dry potassium phosphate based mixture is configured to be mixed with the aqueous solution to thereby form a reabsorbable bio-material slurry, wherein the proteoglycans are between about 1-10 weight percent of the dry composition, and wherein the proteoglycans act as active regulators of collagen fibrillogenesis to thereby structure tissue of a patient by organizing a bone extracellular matrix.

In one particular example, the bio-material composition comprises mono potassium phosphate between about 44-61% of the total composition. The mono potassium phosphate provides faster absorption of the composition. The bio-material composition also comprises calcium sodium phosphosilicate between about 4-9% of the total composition. The calcium sodium phosphosilicate provides additional strength and platform for bone growth and helps provide improved handling characteristics. The bio-material composition also comprises mono sodium phosphate between about 4-9% of the total composition, which helps control ion release. The bio-material composition also comprises magnesium oxide between about 30-45% of the total composition, which may be hard burned to provide controlled reactivity. The MgO enables the composition to be reabsorbed faster compared to existing calcium bone void fillers. The MgO stimulates osteoblast activity as osteoblasts use magnesium as fuel in the bone formation process. The bio-material composition also comprises proteoglycans between about 0-5% of the total composition. The proteoglycans are osteoconductive and osteoinductive because it allows for new bone growth along the bone-implant interface as well as within the bone-implant interface. The bio-material composition also comprises phosphoric acid between about 0-5% of the total composition, which helps break down MgO to generate more phosphate. Various combinations of the above components and percentages are possible as well.

The bio-material composition may be applied to various sites including but not limited to sites on or adjacent to bone; sites on, in, or adjacent to a cartilage; sites in, on, or proximate to bone or cartilage, and bone or cartilage contacting surfaces of implant devices. The bio-material composition may be applied directly to bone defects acting as a bone filler, bone cement, delivery device, bone graft and/or general binder matrix. Alternatively, the bio-material composition may be used in conjunction with various fixation devices such as screws and plates.

In a second aspect, the present disclosure comprises a method for producing a bio-material with increased porosity and reabsorption characteristics, the method comprising: (a) supplying the dry potassium phosphate based mixture of the first aspect, and (b) mixing the dry potassium phosphate based mixture with the aqueous solution of the first aspect to form the reabsorbable bio-material slurry of the first aspect.

In a third aspect, the present disclosure comprises a method for back-filling a bone defect void using a bio-material with increased porosity and reabsorption characteristics, the method comprising: (a) removing a bone defect from a bone to create a void, (b) mixing the dry potassium phosphate based mixture with the aqueous solution of the first aspect to form the reabsorbable bio-material slurry of the first aspect, and (c) back-filling the void with the reabsorbable bio-material slurry, wherein the reabsorbable bio-material slurry is osteoconductive and osteoinductive, thereby enabling new bone growth in the patient along a bone-implant interface as well as within the bone-implant interface.

These as well as other aspects, advantages, and alternatives, will become apparent to those of ordinary skill in the art by reading the following detailed description, with reference where appropriate to the accompanying drawings.

### DETAILED DESCRIPTION

Exemplary devices and systems are described herein. It should be understood that the word “exemplary” is used herein to mean “serving as an example, instance, or illustration.” Any embodiment or feature described herein as “exemplary” is not necessarily to be construed as preferred or advantageous over other embodiments or features. The exemplary embodiments described herein are not meant to be limiting.

As used herein, with respect to measurements, “about” means +/−5%.

As used herein, “Osteoconductive” is the ability of material to serves as a scaffold for viable bone growth and healing.

As used herein, “Osteoinductive” refers to the capacity to stimulate or induce bone growth.

As used herein, “Biocompatible” refers to a material that does not elicit a significant undesirable response in the recipient.

As used herein, “Bioresorbable” is defined as a material's ability to be resorbed in-vivo through bodily processes. The resorbed material may be used the recipients body or may be excreted.

### I. Preparing/Supplying the Dry Mixture

The dry mixture of the present disclosure generally comprises: MgO, monobasic potassium phosphate, monobasic sodium phosphate, proteoglycans, calcium sodium phosphosilicate, and an antibiotic, wherein a weight percent ratio of monobasic potassium phosphate to MgO is between about 3:1 and 1:1. It may be preferable to produce the dry mixture in advance. After it is prepared it should be stored in a sterile environment and more preferably a sterile and sealed container or packaging.

The dry components of the mixture can be mixed using a variety of methods including hand mixing or machine mixing. One method for mixing, sizing, and homogenizing the various powders is via vibratory milling. Another homogenization method utilizes a ribbon mixer wherein the particles are ground to a fine size. It may be preferable to mix the dry components again on-site before the addition of the activating aqueous solution.

The MgO of the composition is optionally subjected to a calcination and thermal decomposition process. Calcination of the MgO is a treatment process in the absence or limited supply of air or oxygen applied to ores and other solid materials to bring about a thermal decomposition. Thermal decomposition, or thermolysis, is a chemical decomposition caused by heat. The decomposition temperature of a substance is the temperature at which the substance chemically decomposes. The reaction is usually endothermic as heat is required to break chemical bonds in the compound undergoing decomposition. In other words, this process allows the MgO to break down and turn into a hydrate so it will be reabsorbed by the body.

Calcination durations and temperatures are determined empirically, depending on the final characteristics and setting times desired. In some embodiments calcination temperatures of up to about 1300° C. for up to several hours are used, although calcination can be varied. Those of ordinary skill in the art of preparation of similar bone compositions could routinely determine the appropriate calcination conditions to achieve the desired properties.

In addition to the aqueous forms, the composition of the present disclosure can be a gel comprising the dry mixture.

Generally, pharmaceutical grade compounds are utilized when available. Sterilization of the components, utensils, solutions, etc., used to make and apply the slurry may be required using suitable sterilization techniques known in the art including but not limited to chemical sterilization techniques, such as gassing with ethylene oxide, and sterilization by means of high-energy radiation, usually γ radiation or β radiation.

While the formulations described herein are the preferred proportions, a range of dry constituents can also be used. For example, a suitable range for the mono potassium phosphate is generally between about 20-70 weight percent, preferably between about 40-65 weight percent. In some situations and/or embodiments it is preferable to use the potassium phosphate at a range between about 40-50 weight.

A suitable range for the magnesia (i.e., MgO) is generally between about 10-60, preferably between 10-50, and even more preferably between 30-50 weight percent. In some situations and/or embodiments between about 35 and 50 weight percent can be used.

Calcium sodium phosphosilicate is preferably added at about 1-15 weight percent, more preferably between about 1-10 weight percent. In one particular example, the calcium sodium phosphosilicate is added between about 4-9% of the total composition. Higher percentages can be employed in certain situations. As examples, the calcium sodium phosphosilicate may comprise 45S5 bioactive glass fibers, silica bioactive glass, Silicon dioxide, Silicate, Calcium oxide, Sodium oxide, Phosphorus pentoxide, and combinations thereof.

Mono sodium phosphate is preferably added at about 1-15 weight percent, more preferably between about 1-10 weight percent. In one particular example, the mono sodium phosphate is added between about 4-9% of the total composition. Higher percentages can be employed in certain situations.

Proteoglycans are generally present at weight percent between 0.5 and 20, preferably about 0.5-10 weight percent of the dry composition. As examples, the proteoglycans comprise between about 1-5 weight percent of the dry composition, between about 1.5-2.5 weight percent of the dry composition, between about 1.5-3 weight percent of the dry composition, between about 1.5-3.5 weight percent of the dry composition, between about 1.5-4 weight percent of the dry composition, between about 1.5-4.5 weight percent of the dry composition, about 2 weight percent of the dry composition, about 1.5-2 weight percent of the dry composition, or about 1.5-2.5 weight percent of the dry composition. Suitable proteoglycans include mineral-collagen composite matrix, fibers, granules, morcellized fibers, nanoparticles, and combinations thereof.

Typically an antibiotic, antibacterial or antiviral agent is added at a weight percent of less than about 20 weight percent of the dry composition, preferably between about 0.5 and 10 weight percent, more preferably between about 1 and 5 weight percent. Any antibiotics typically used in joint replacement and repair surgeries can be used.

In another embodiment, the dry mixture of the present disclosure generally comprises monopotassium phosphate (KH₂PO₄) (about 44 weight percent), MgO (about 41 weight percent), calcium hydroxylapatite (Ca₅(PO₄)₃OH₂) (about 8 weight percent), monosodium phosphate (NaH₂PO₄) (about 3 weight percent), and sucrose (C₁₂H₂₂O₁₁) (about 4 weight percent).

### II. Solution Formulation

Water (or another aqueous solution) can be added in a large range of weight percents generally ranging from about 15-40 weight percent, preferably between about 20-35 weight percent and even more preferably between about 28-32 weight percent. It was found that a saline solution may be used. An exemplary saline solution is a 0.9% saline solution. As another example, the aqueous solution comprises blood (e.g., the blood from the patient or blood other than that of the patient). In yet another example, the aqueous solution comprises sodium chloride (about 0.75 weight percent), monosodium phosphate (NaH₂PO₄) (about 16.60 weight percent), and water (about 82.65 weight percent). Other aqueous solutions are possible as well.

### III. Forming a Reabsorbable Bio-Material Slurry

The dry mixture is preferably activated on-site. Activation comprises mixing the dry composition with an aqueous solution (such as in a sterile mixing vessel to form a reabsorbable bio-material slurry). Water (e.g., sterile water (or other sterile aqueous solution, e.g., i.e., slight saline solution) is generally added up to about 40% of the dry weight, although the amount of water can be adjusted to form a bio-material of varying viscosity. In one embodiment, the mixing vessel and any utensils are sterilized prior to use. Various mixing vessels can be used including but not limited to a sterile medicine cup, bowl, dish, basin or other sterile container. In another example, the aqueous solution comprises blood (e.g., the blood from the patient or blood other than that of the patient).

Mixing can be achieved by a variety of techniques used in the art including hand and electric/automated mixing. One preferred method is to hand mix with a sterile spatula or other mixture utensil. The reabsorbable bio-material slurry is typically hand mixed for between about 1-10 minutes, although mixing times can be adjusted depending upon conditions and mixing means.

It is possible to mix the slimy using manual hand mixers like the Mixevac III from Stryker (Kalamzoo, Mich.) or an electric bone mixer like the Cemex Automatic Mixer from Exactech (Gainesville, Fla.).

The reabsorbable bio-material slurry can be created in injectable, paste, puddy and other forms. Because the slurry is produced at the user site, the consistency of the material can be manipulated by varying the amount of water added to the dry mixture. Increasing the water content generally increases the flowability while decreasing the water content tends to thicken the slurry.

Working times can be increased or decreased by varying the temperatures of bio-material components. Higher temperature components tend to react and set quicker than cooler components. Thus regulating the temperature of the water (or other reactants) can be an effective way to regulate working time.

The inventor has found that the use of a phosphoric acid solution instead of water increases the bonding strength of the material. The molarity of the phosphoric acid can vary, as long as the eventual pH of the slurry is not hazardous to the patient, or contraindicative to healing.

### IV. Applying the Reabsorbable Bio-Material Slurry to the Site

Once the reabsorbable bio-material slurry has been formed it is applied to (and optionally also around) the site of desired cartilage growth. The slurry can be applied to the site in a number of ways including but not limited to spreading an amount of the material to the site using a sterile spatula, tongue blade, knife or other sterile implement useful for spreading a paste or paddy-like material. In some situations it may be preferable to use a relatively thick consistency like a paste or paddy when applying the activated slurry, since such consistencies tend to stick to bone and other surface more easily than thinner ones. If an injectable formation is desired, it can be applied using a syringe or other similar device.

### V. Proteoglycans

Proteoglycans can be used in compositions of the present disclosure as it provides a role in bone tissue formation by promoting collagen fibers consolidation and their connection with crystals of minerals.

### VI. Calcium Sodium Phosphosilicate

Calcium sodium phosphosilicate can be used in compositions of the present disclosure as it provides bioactive glass material that degrades in the aqueous oral environment to release calcium and phosphate ions, leading to bone formation.

Surprisingly and unexpectedly, it was discovered that the compositions and methods of the present disclosure provide improved reabsorption, improved porosity, and improved cohesion. This result was particularly surprising given recent studies showing the inability of calcium phosphate cements to be reabsorbed. The phosphate component of the composition allows for increased porosity. The increased porosity allows for a scaffold which provides a suitable microenvironment for the incorporation of cells or growth factors to regenerate damaged tissues and bony ingrowth. Scaffolds are generally highly porous with interconnected pore networks to facilitate nutrient and oxygen diffusion and waste removal. This scaffolding will also help with absorbability. The sugar component of the composition allows for adhesive properties. The adhesive properties are desired since the placement of this product is in bone void of some size. The adhesive qualities will allow the product to attach itself to both sides and create a scaffold to allow cells to regenerate bone.

### VII. Antibiotics

Various antibiotics or other antibacterial and anti-viral compositions and agents can be added to the composition. The invented bio-material can act as a delivery device or the antibiotics can be added to protect against bacterial infection during surgery.

Cationic antibiotics, especially aminoglycosides and certain peptide antibiotics may be most desirable when incorporating drugs into the bio-material. Suitable aminoglycosides include but are not limited to: amikacin, butirosin, dideoxykanamycin, fortimycin, gentamycin, kanamycin, lividomycin, neomycin, netilmicin, ribostamycin, sagamycin, seldomycin and epimers thereof, sisomycin, sorbistin, spectinomycin and tobramycin. Using inorganic salts like sulfates, phosphates, hydrogenphosphates may be preferable, sulfates being the most preferable. Further information about using antibiotics and growth factors in bio-materials can be found in U.S. Pat. No. 6,485,754, issued to Wenz, which is hereby incorporated by reference in its entirety. Growth factors include but are not limited to growth factors like transforming growth factor TGF-β. Vancomycin and similar antibiotics can also be used.

### VIII. Poloxamines

Various poloxamines (i.e., X-shaped poly(ethylene oxide)-poly(propylene oxide) block copolymers with an ethylenediamine core (such as Tetronic®) can be added to the composition. As non-limiting examples, Tetronic® 304, 904, 90R4, 908, 1107, 1301 and 1307 can be added to the composition.

The presence of a poloxamine (such as Tetronic®) can improve hydrolysis, setting reaction, microstructure, and mechanical properties of the composition. In particular, the presence of a poloxamine ion the composition may prolong the setting time of the reabsorbable bio-material slurry. Additional or alternatively, the presence of a poloxamine in the composition may increase the compressive strength of the cured composition.

### IX. Bone Graft Material

In one embodiment the composition of present disclosure provides a bone substitute and a platform for bone formation. An advantage of the substance is its gradual absorption by the body without rejection or reaction to contacted structures. A further advantage of the invented composition is its significant osteoproliferative properties. In fact, we have conducted studies that demonstrated that the composition of the present disclosure enhanced bone formation to such a surprising degree that it appears that the composition is also osteoinductive, which is completely unexpected and unprecedented for a multi-purpose biomaterial without the use of growth factors. The bio-material is also believed to have micro and macro pores. Unexpectedly, initial tests have shown that the invented composition is capable of promoting motion preservation in a bone.

We have also observed that compositions of the present disclosure have unique bonding characteristics suitable for fixation of various medical prosthesis.

### X. Additional Embodiments

The formulations disclosed herein may incorporate additional fillers, additives and supplementary materials. The supplementary materials may be added to the bio-material in varying amounts and in a variety of physical forms, dependent upon the anticipated use. The supplementary materials can be used to alter the bio-material in various ways.

Supplementary materials, additives, and fillers are preferably biocompatible and/or bioresorbable. In some cases it may be desirous for the material to be osteoconductive and/or osteoinductive as well. Suitable biocompatible supplementary materials include but are not limited to: bioactive glass compositions, calcium sulfates, coralline, polyatic polymers, peptides, fatty acids, collagen, glycogen, chitin, celluloses, starch, keratins, nucleic acids, glucosamine, chondroitin, and denatured and/or demineralized bone matrices, and other materials, agents, and grafts (autografts, allografts, xenografts). Other suitable supplementary materials are disclosed in U.S. Pat. No. 6,331,312 issued to Lee and U.S. Pat. No. 6,719,992 issued to Constanz, which are hereby incorporated by reference in their entireties.

In another embodiment of the present disclosure the bio-material contains a radiographic material which allows for the imaging of the material in vivo. Suitable radiographic materials include but are not limited to barium oxide and titanium.

In yet another embodiment the invented bio-material contains a setting retarder or accelerant to regulate the setting time of the composition. Setting regulators are preferable biocompatible. Suitable retarders include but are not limited to sodium chloride, sodium fluosilicate, polyphosphate sodium, borate, boric acid, boric acid ester and combination thereof.

The disclosed bio-material may also be prepared with varying degrees of porosity. Controlling porosity can be accomplished through a variety of means including: controlling the particle size of the dry reactants, and chemical and physical etching and leaching. The biomaterial may be used as delivery system by incorporating biologically active compounds into the bio-material (i.e., antibiotics, growth factors, cells, etc.). A porous bio-adhesive increases the effectiveness of such a delivery system.

The disclosed bio-material composition may also be seeded with various living cells or cell lines. Any known method for harvesting, maintaining and preparing cells may be employed. See U.S. Pat. No. 6,719,993 issued to Constanz, U.S. Pat. No. 6,585,992 issued to Pugh and, U.S. Pat. No. 6,544,290 issued to Lee.

We have shown that compositions of the present disclosure are extremely useful as a scaffold for hard tissue growth and possibly soft tissue growth as well. In addition, tissue-producing and tissue-degrading cells may be added to the composition included but not limited to: osteocytes, osteoblasts, osteoclasts, chondrocytes, fibroblasts, cartilage producing cells, and stern cells. Methods of isolating and culturing such cells are well known in the art.

The composition of the present disclosure can incorporated into an orthopedic kit comprising the material (i.e., monobasic potassium phosphate, metal oxide, calcium containing compounds etc.) in dry form, an activator solution (water or other aqueous solution), and any medical devices (i.e., syringes, knives, mixing materials, spatulas, etc.), implants, or other agents needed during an operation using the invented composition. The material and activator solution will preferably be present in a predetermined, optimized ratio. Other embodiments of such an orthopedic kit can also be envisioned. The biomaterial and other kit components are preferably sterilized by techniques well known in the art.

### XL. Example Method

A method for back-filling a bone defect void using a bio-material with increased porosity and reabsorption characteristics is described herein. The method includes (a) removing a bone defect from a bone to create a void, (b) mixing a dry potassium phosphate based mixture with an aqueous solution to form a reabsorbable bio-material slurry, wherein the dry potassium phosphate based mixture comprises MgO, monobasic potassium phosphate, monobasic sodium phosphate, proteoglycans, calcium sodium phosphosilicate, and an antibiotic, wherein a weight percent ratio of monobasic potassium phosphate to MgO is between about 3:1 and 1:1, wherein the dry potassium phosphate based mixture is configured to be mixed with the aqueous solution to thereby form a reabsorbable bio-material slurry, wherein the proteoglycans are between about 1-10 weight percent of the dry composition, and (c) back-filling the void with the reabsorbable bio-material slurry, wherein the reabsorbable bio-material slurry increases osteoblast activity in the bone to help maintain the structure of the bone.

In such a method, the reabsorbable bio-material slurry turns to bone to provide improved bone structure in the bone. In contrast, traditional calcium-based bone fillers provide a scaffolding on which bone can grow, but do not turn into bone like the above-described composition. As such, the osteocytes in traditional calcium-based bone fillers run out and the bone filler deteriorates and is reabsorbed into the body. The advantage of the reabsorbable bio-material slurry described herein is that it actually turns into bone to thereby provide improved bone structure. In addition, the reabsorbable bio-material slurry described herein increases osteoblast activity in the bone due to the magnesium present in the reabsorbable bio-material slurry. Osteoblasts are the major cellular component of bone. Osteoblasts are specialized, terminally differentiated products of mesenchymal stem cells. They synthesize dense, crosslinked collagen and specialized proteins in much smaller quantities, including osteocalcin and osteopontin, which compose the organic matrix of bone. As such, the above method comprises a method for preserving bone comprising stimulating osteoblasts due to the magnesium present in the reabsorbable bio-material slurry to help maintain bone structure.

As discussed above, the method includes removing a bone defect from a bone to create a void. The bone defect may take a variety of forms. In particular, the bone defect may be selected from a group consisting of: a bone cyst, a bone marrow lesion, and an osteoporotic bone. A bone cyst is a fluid-filled hole that develops inside a bone. They mostly occur in children and young adults. Bone cysts do not usually cause any symptoms, they are not cancerous and they do not usually pose a serious threat to health. Bone marrow lesions (BMLs) or using older terminology “bone marrow edema” is characterized by excessive water signals in the marrow space on magnetic resonance imaging or ultrasound; BMLs constitute a central component of a wide variety of inflammatory and non-inflammatory rheumatologic conditions affecting the musculoskeletal system: BMLs are not only considered significant sources of pain but also linked to increased disease activity in many musculoskeletal conditions (for example, osteoarthritis, rheumatoid arthritis). The bone defects of the above method may be defects of the extremities and/or pelvic bone, as specific examples.

In one example, the method further includes positioning an anchor in the void prior to back-filling the void with the reabsorbable bio-material slurry. Such an anchor may provide additional structural support for the bone. The anchor may be a reabsorbable polymer material or a metal material. One example polymer material is poly-l d-lactide (PLDLA).

### XII. Conclusion

Having described the basic concept of the present disclosure, it will be apparent to those skilled in the art that the foregoing detailed disclosure is intended to be presented by way of example only, and is not limiting. Various alterations, improvements, and modifications are intended to be suggested and are within the scope and spirit of the present disclosure. Additionally, the recited order of the elements or sequences, or the use of numbers, letters or other designations therefore, is not intended to limit the claimed processes to any order except as may be specified in the claims. Accordingly, the present disclosure is limited only by the following claims and equivalents thereto.

All publications and patent documents cited in this application are incorporated by reference in their entirety for all purposes to the same extent as if each individual publication or patent document were so individually denoted and to the extent they are not inconsistent with the express teachings herein.

What is claimed is:

1. A bio-material composition, comprising:
a dry potassium phosphate based mixture comprising: MgO, monobasic potassium phosphate, monobasic sodium phosphate, proteoglycans, calcium sodium phosphosilicate, and an antibiotic, wherein a weight percent ratio of monobasic potassium phosphate to MgO is between about 3:1 and 1:1, wherein the dry potassium phosphate based mixture is configured to be mixed with the aqueous solution to thereby form a reabsorbable bio-material slurry, wherein the proteoglycans are between about 1-10 weight percent of the dry composition, and wherein the proteoglycans act as active regulators of collagen fibrillogenesis to thereby structure tissue of a patient by organizing a bone extracellular matrix.

2. The bio-material composition of claim 1, wherein the proteoglycans are selected from the group consisting of: mineral-collagen composite matrix, fibers, granules, morcellized fibers, nanoparticles, and combinations thereof.

3. The bio-material composition of claim 1, wherein the calcium sodium phosphosilicate is selected from a group consisting of: 45S5 bioactive glass fibers, silica bioactive glass, Silicon dioxide, Silicate, Calcium oxide, Sodium oxide, Phosphorus pentoxide, and combinations thereof.

4. The bio-material composition of claim 1, wherein the proteoglycans are between about 1-5 weight percent of the dry composition.

5. The bio-material composition of claim 1, wherein the proteoglycans are between about 1.5-2.5 weight percent of the dry composition.

6. The bio-material composition of claim 1, wherein the proteoglycans are between about 1.5-3 weight percent of the dry composition.

7. The bio-material composition of claim 1, wherein the proteoglycans are between about 1.5-3.5 weight percent of the dry composition.

8. The bio-material composition of claim 1, wherein the proteoglycans are between about 1.5-4 weight percent of the dry composition.

9. The bio-material composition of claim 1, wherein the proteoglycans are between about 1.5-4.5 weight percent of the dry composition.

10. The bio-material composition of claim 1, wherein the proteoglycans are about 2 weight percent of the dry composition.

11. The bio-material composition of claim 1, wherein the proteoglycans are about 1.5-2 weight percent of the dry composition.

12. The bio-material composition of claim 1, wherein the proteoglycans are about 1.5-2.5 weight percent of the dry composition.

13. The bio-material composition of claim 1, wherein the bio-material composition is osteoconductive and osteoinductive, thereby enabling new bone growth in the patient along a bone-implant interface as well as within the bone-implant interface.

14. The bio-material composition of claim 1, wherein the antibiotic is between about 1-5 percent of the dry composition.

15. The bio-material composition of claim 1, wherein the antibiotic is an aminoglycoside antibiotic.

16. A method for producing a bio-material with increased porosity and reabsorption characteristics, the method comprising:
supplying the dry potassium phosphate based mixture of claim 1; and
mixing the dry potassium phosphate based mixture with the aqueous solution of claim 1 to form the reabsorbable bio-material slurry of claim 1.

17. The method of claim 16, wherein the proteoglycans are between about 1-10 weight percent of the dry composition.

18. A method for back-filling a bone defect void using a bio-material with increased porosity and reabsorption characteristics, the method comprising:
removing a bone defect from a bone to create a void;
mixing the dry potassium phosphate based mixture of claim 1 with the aqueous solution of claim 1 to form the reabsorbable bio-material slurry of claim 1; and
back-filling the void with the reabsorbable bio-material slurry, wherein the reabsorbable bio-material slurry is osteoconductive and osteoinductive, thereby enabling new bone growth in the patient along a bone-implant interface as well as within the bone-implant interface.

19. The method of claim 18, wherein the reabsorbable bio-material slurry turns to bone to provide bone structure in the bone.

20. The method of claim 18, wherein the bone defect is selected from a group consisting of: a bone cyst, a bone marrow lesion, and an osteoporotic bone.

21. The method of claim 18, further comprising positioning an anchor in the void prior to back-filling the void with the reabsorbable bio-material slurry, wherein the anchor provides additional structural support for the bone.

22. The method of claim 21, wherein the anchor comprises a polymer or a metal.
